(19) United States
(12) Patent Application Publication
(10) Pub. No.: US 2005/0181025 A1
(43) Pub. Date: Aug. 18, 2005

(54) Preparation for wound healing and prevention of bandage adhesion to the wound

(76) Inventors: Vladimir Velebny, Usti nad Orlici (CZ); Lubos Sobotka, Hradec Kralove (CZ); Stanislav Pavek, Sloupnice (CZ); Jana Ruzickova, Kromeriz (CZ)

Correspondence Address: WOOD, HERRON & EVANS, LLP, 2700 CAREW TOWER, 441 VINE STREET, CINCINNATI, OH, 45202, US

(21) Appl. No.: 10/501,887

(22) PCT Filed: Jan. 15, 2003
(86) PCT No.: PCT/CZ03/00003

(30) Foreign Application Priority Data
Jan. 18, 2002 (CZ) PUV02-12746

(51) Int. Cl. A61K 31/728; A61L 15/00

(52) U.S. Cl. 424/445; 514/54

(57) ABSTRACT

Preparation for wound healing and prevention of adhesion to the wound containing a physiologically acceptable salt of hyaluronic acid, iodine and potassium iodine.

### FIELD OF THE INVENTION

The present invention relates to a preparation based on a pharmacologically acceptable salt of hyaluronic acid which is applied to the wound and it is able to prevent adhesion of the bandage to the wound and at the same time to speed up the process of healing.

### BACKGROUND

The present state of healing of acute and chronic complicated wounds is based on the use of a whole range of different materials and techniques. Of course the ideal solution is to clean the wound and then take care of it by a surgical treatment (suture, skin auto-grafting, etc.). However said method of treatment is possible only for a very small number of skin defects and it applies to surgical wounds and acute non-infected wounds.

Most of the large acute wounds are infected and they contain necrotic parts. The bacterial contamination can be supposed for chronic wounds and practically in all cases there are some necroses. These large subacute and chronic wounds are very difficult to treat.

At present the following systems described below are used for healing of large or chronic wounds.

One of the systems is a repeated re-bandage with moistened gauzes which assure a permanent damp environment for wound healing. The gauze is moistened by a physiological solution or a solution containing an antiseptic (Betadine, chloramine, rivanol, etc.). Said method is difficult since it requires a frequent re-bandage every 4 to 6 hours. This method also does not lead to the complete sterilization of the wound and it does not prevent a maceration of skin in the wound surroundings.

Since bandages which are not regularly moistened very often stick to the wound, they are often saturated by Vaseline. This is the so-called greasy gauze. The vaseline is often combined with iodine so that the bandage has antimicrobial properties. A disadvantage of such use is that the vaseline closes the wound and all the necrotic parts and infection accumulate under the bandage. Problems also arise with removing vaseline from deeper wounds during re-bandaging. For this reason this method is used only for superficial wounds.

Smaller deep wounds are often covered by a plastic bandage which maintains a moist environment in the wound. This bandage comprises cellulose derivates and sometimes also contains a seaweed extract (alginate). Such bandage meets mainly the covering function since it does not have disinfection effects on the wound and the bandage itself does not contribute to the elimination of necrotic tissue. There are very complicated and expensive systems available on the market which use, for example, collagenase or papaine in the ointment or cream base for the elimination of necrotic tissue. Tissue secretion is removed mainly by preparations containing active coal.

If the necrotic parts are removed and the wound is disinfected, then preparations providing a hydrated environment which should be beneficial for wound healing are used. This is due mainly to the use of alginate, a polysaccharide, which is produced by seaweeds. Some companies use carboxymethylcellulose for ensuring hydration of the wound environment, which is essential for its healing. A disadvantage of this system is that it requires cleaning of the wound in a separate step prior to the application of the substances enhancing wound healing and, moreover, it is not able to keep the wound sterile and prevent the later development of infection without further support.

Different growth factors and the sodium salt of hyaluronic acid are used in some preparations for enhancing healing effects. A disadvantage of the system in this case is that it also requires cleaning of the wound in a separate step prior to the application of substances enhancing wound healing. Moreover, said preparations are not able to keep the wound sterile without further support and prevent infection development.

A combination of collagen and a chemically modified cellulose is also used for therapy of chronic wounds. If this system is not completed by other substances it is not able to ensure wound disinfection, removing of secretion, etc. on its own.

Furthermore, some authors recommend the use of oxygenotherapy in hyperbaric chambers for healing of chronic wounds (especially for diabetic patients). However this therapy is very demanding concerning the apparatus.

A vacuum-therapy is a kind of treatment in which a porous elastic sponge is applied to the wound and the wound is covered by an impermeable sheet. The air is then exhausted from the wound and the incurred under pressure should ensure cleaning of the wound and secretion exhausting. The sponge is exchanged in regular intervals. This is very apparatus demanding and it is suitable only for highly specialized workplaces, and moreover, it is limited for selected diagnoses only.

### SUMMARY OF THE INVENTION

One embodiment is a method to enhance wound healing by providing to a wound a composition of a physiologically acceptable salt of hyaluranic acid and an iodine complex under conditions (e.g., for a sufficient duration) sufficient to enhance healing of the wound. The iodine complex comprises a solution of iodine and potassium iodine. The composition may be applied to the wound directly or indirectly (e.g., on a wound-contacting portion of a bandage), and minimizes adherence of the wound-contacting portion to the wound. The composition disinfects the wound and reduces further infection, removes wound secretions thereby reducing wound maceration, maintains wound hydration, and enhances granulation and epithelial cell formation of wounded tissue.

Another embodiment is a method of minimizing or preventing adhesion to a wound of a wound-contacting surface of a bandage or other wound covering. A hyaluranic acid, iodine, and potassium iodine composition is provided to a wound and covered, or the composition is applied to the wound covering, which is then applied to the wound. The treated surface of the covering reduces or prevents adhesion to the wound.

Another embodiment is a method to enhance wound healing by providing a wound with a biocompatible hyaluranic acid, iodine, and potassium iodine composition for a sufficient duration to enhance wound healing. The wound can then be covered. Alternatively, the composition can be provided on a wound-contacting surface of a bandage or other wound covering. The wound can be monitored as it heals. Iodine and potassium iodine in the composition disinfect the wound.

Another embodiment is a composition of a physiologically acceptable formulation of iodine, potassium iodine, and hyaluranic acid for wound healing. The composition may be formulated as a solution or a gel, and may be applied directly or indirectly to wounded tissue. Iodine may be at a concentration ranging from 0.05 to 2.5% by weight of the composition and potassium iodine may be at a concentration ranging from 0.05 to 5% by weight of the composition. The hyaluranic acid may be 0.05 to 10% by weight of the composition. It may have a molecular weight ranging from 200,000 to 2,500000, and may be a salt of sodium, potassium, lithium, calcium, magnesium, zinc, cobalt, or manganese.

These and other embodiments are disclosed in the following detailed description.

### DETAILED DESCRIPTION

The aim of the invention is to create a preparation, the application of which to the wound would provide an environment that prevents wound infections and disinfects the wound at the same time. Furthermore, it would exhaust the secretion from the wound and thus prevent the maceration of the wound and its surroundings and maintain a good hydration in the wound and the presence of tissue mediators and enzymes. The preparation should also ensure an ideal environment needed for the formation of granulation tissue and other regeneration processes in the wound. It should prevent bandage adhesion to the wound, protect the wound surroundings and enable monitoring of possible wound changes (especially development of bleeding).

The disadvantages stated in the background of the invention and the aims laid out above are solved by the preparation for wound healing and prevention of bandage adhesion to the wound according to the invention. The subject-matter of the invention is a preparation containing physiologically acceptable salt of hyaluronic acid having the molecular weight in the range from 200,000 to 2,500,000 in gel or solution together with iodine and potassium iodine.

A physiologically acceptable salt of hyaluronic acid is selected from a group containing sodium salt, potassium salt, lithium salt, calcium salt, magnesium salt, zinc salt, cobalt salt, manganese salt or a combination thereof The preparation according to the invention may be in the form of viscous aqueous solution or gel.

The preparation according to the invention may contain a physiologically acceptable salt of hyaluronic acid in the concentration from 0.05 to 10% by weight, iodine in the concentration from 0.05 to 2.5% by weight, and potassium iodine in the concentration from 0.05 to 5% by weight as substances with antiseptic properties acting bacteriostatically and fungistatically.

One embodiment of the invention is a preparation containing a physiologically acceptable salt of hyaluronic acid in the concentration from 0.05 to 10.0% by weight, iodine in the concentration from 0.075 to 1% by weight, and potassium iodine in the concentration from 0.075 to 1% by weight.

The preparation according to the invention is prepared by dissolving the above mentioned substances in sterile water.

The preparation for wound healing and prevention of bandage adhesion to the wound is applied either directly to the wound or is spread in the needed amount on that side of a bandage which is then placed on the wound.

A combination of suitable salts of hyaluronic acid with iodine and potassium iodine is itself able to satisfy the above mentioned conditions. Salts of hyaluronic acid belong to the most hydrophilic molecules in nature. The preparation ensures the secretion of tissue fluid after its application on the gauze and the wound and also a constantly damp environment. In a combination with iodine and potassium iodine it disinfects the wound for a short time which provides a clean environment in the wound. The salts of hyaluronic acid have also a strong healing effect, they act very positively during all phases of the healing process. This all has a positive effect on the formation of granulation tissue and the following epithelisation and thereby the healing of the wound. The advantage is also the possibility of bandage monitoring and the fact that only the wound itself is hydrated and the skin around the wound is intact.

The preparation according to the invention activates keratinocytes to produce cytokines in contrary to iodine and potassium iodine separately (an iodine complex) and hyaluronan separately. The cytokines produced are the activators and chemoatractants for different cells of white line which shows up in a speeded wound cleaning and a preparation of the wound surface for the formation of granulation tissue. Furthermore, they activate keratinocytes which allows the ingrowth of the wound. The above mentioned unexpected effects are not exhibited by either one of the three components of the preparation according to the invention if applied separately. Iodine in combination with another oligomer or polymer substances is used in some preparations (e.g. Betadine). In our case, it is not possible to use the combination of hyaluronane as a polymer substance and iodine directly since it is not possible to reach the required concentration of iodine in solution. For this reason potassium iodine is added forming the iodine complex. The iodine complex has the requested solubility in water as well as the combination of iodine and potassium iodine is more acceptable for the cells than the iodine alone.

### EXAMPLE 1

0.1 g of iodine is dissolved in the solution of 0.15 g of potassium iodine in 50 ml of sterile water for injection. Furthermore, 1.5 g of sodium hyaluronate having the molecular weight 1,000,000 is dissolved in 50 ml of sterile water for injection. The solutions are prepared separately and they are separately sterilized. They are mixed together under sterile conditions after sterilization. The highly viscous solution that is produced can be applied directly to the wound which is afterwards covered by the bandage or it can be applied to the bandage which is afterwards placed on the wound.

### EXAMPLE 2

1.0 g of iodine is dissolved in the solution of 1.5 g of potassium iodine in 50 ml of sterile water for injection. Furthermore, 1.5 g of sodium hyaluronate having the molecular weight 1,500,000 is dissolved in 50 ml of sterile water for injections. The solutions are prepared separately and they are separately sterilized. They are mixed together under sterile conditions after sterilization. The highly viscous solution which is produced can be applied directly to the wound which is covered afterwards by the bandage or it can be applied to the bandage, which is afterwards placed on the wound.

### EXAMPLE 3

0.5 g of iodine is dissolved in the solution of 0.75 g of potassium iodine in 50 ml of sterile water for injection. The gel of hyaluronate having the molecular weight 1,500,000 is produced by mixing 2 g of hyaluronan with 50 ml of water for injection in a separate flask. The solution and the gel are prepared separately and they are also separately sterilized. They are mixed together under sterile conditions after sterilization. It is possible to apply the produced gel in a thin layer directly to the wound which is afterwards covered by the bandage.

Other variations or embodiments of the invention will also be apparent to one of ordinary skill in the art from the above descriptions. Thus, the forgoing embodiments are not to be construed as limiting the scope of the following claims.

1. A composition for wound healing and prevention of adhesion to the wound comprising a physiologically acceptable salt of hyaluronic acid having molecular weight from 200,000 to 2,500,000, iodine and potassium iodine, forming a composition for would healing and preventing adhesion of a would covering to a wound.

2. The composition of claim 1 wherein said physiologically acceptable salt of hyaluronic acid is selected from at least one of a sodium salt, potassium salt, lithium salt, calcium salt, magnesium salt, zinc salt, cobalt salt or manganese salt.

3. The composition of claim 1 wherein the concentration of the physiologically acceptable salt of hyaluronic acid is in the range from 0.05 to 10.0% by weight, the concentration of iodine is in the range from 0.05 to 2.5% by weight, and the concentration of potassium iodine is in the range from 0.05 to 5% by weight.

4. The composition of claim 3 wherein the concentration of the physiologically acceptable salt of hyaluronic acid is in the range from 0.05 to 10.0% by weight, the concentration of iodine is in the range from 0.075 to 1% by weight, and the concentration of potassium iodine is in the range from 0.075 to 1% by weight.

5. The composition of claim 1 in the form of a sterile aqueous solution or a gel.

6. A method to enhance wound healing comprising providing to a wound a composition comprising a physiologically acceptable salt of hyaluranic acid and an iodine complex under conditions sufficient to enhance healing of the wound.

7. The method of claim 6 wherein the iodine complex comprises a solution of iodine and potassium iodine.

8. The method of claim 6 wherein the composition is provided directly to the wound.

9. The method of claim 6 wherein the composition is provided indirectly to the wound.

10. The method of claim 6 wherein the composition is provided to a wound-contacting portion of a wound covering.

11. The method of claim 10 wherein the covering is a bandage or a gauze.

12. The method of claim 11 further minimizing adherence of the wound-contacting portion to the wound.

13. The method of claim 6 disinfecting and reducing wound infection.

14. The method of claim 6 removing wound secretions thereby reducing wound maceration.

15. The method of claim 6 maintaining wound hydration.

16. The method of claim 6 enhancing granulation of wounded tissue.

17. The method of claim 6 enhancing epithelial cell formation of wounded tissue

18. A method of minimizing wound adhesion of a wound-contacting surface of a wound covering, the method comprising providing a composition comprising hyaluranic acid, iodine, and potassium iodine and providing a covering to the wound, the covering having a wound-contacting surface with minimized wound adhesion.

19. The method of claim 18 providing the composition to the wound and thereafter providing the covering.

20. The method of claim 18 providing the composition to the wound-contacting surface of the covering to form a treated covering, and thereafter providing the treated covering to the wound.

21. The method of claim 18 wherein the covering is a bandage or a gauze.

22. A method to enhance wound healing comprising providing to a wound a biocompatible composition comprising hyaluranic acid, iodine, and potassium iodine for a sufficient duration to enhance wound healing.

23. The method of claim 22 wherein the composition is provided on a wound-contacting surface of a wound covering.

24. The method of claim 22 further comprising covering the wound.

25. The method of claim 22 further comprising intermittently monitoring the wound over the duration.

26. The method of claim 22 wherein the iodine and potassium iodine disinfect the wound.

27. A composition to enhance wound healing, the composition comprising a physiologically acceptable formulation of iodine, potassium iodine, and hyaluranic acid.

28. The composition of claim 27 comprising iodine at a concentration ranging from 0.05 to 2.5% by weight of the composition and potassium iodine at a concentration ranging from 0.05 to 5% by weight of the composition.

29. The composition of claim 27 wherein iodine is added to a potassium iodine solution to form an iodine solution for combining with a hyaluronate solution.

30. The composition of claim 27 wherein hyaluranic acid has a molecular weight ranging from 200,000 to 2,500,000.

31. The composition of claim 27 formulated as at least one of a solution or a gel.

32. The composition of claim 27 where the hyaluranic acid salt is at least one of sodium, potassium, lithium, calcium, magnesium, zinc, cobalt, or manganese.

33. The composition of claim 27 wherein hyaluranic acid salt is 0.05 to 10% by weight of the composition.

34. The composition of claim 27 wherein hyaluranic acid salt is 0.05 to 10% by weight, iodine is 0.075 to 1% by weight, and potassium iodine is 0.075 to 1% by weight.

35. The composition of claim 27 prepared by combining an iodine solution with a hyaluranic acid solution.

36. The composition of claim 27 wherein iodine is dissolved in an aqueous solution of potassium iodine to form the iodine solution.

37. The composition of claim 27 being viscous.
